(19) United States
(12) Patent Application Publication
(10) Pub. No.: US 2021/0343389 A1
(43) Pub. Date: Nov. 4, 2021

(54) SYSTEMS AND METHODS OF PAIN TREATMENT

(71) Applicant: LUCINE, BORDEAUX (FR)

(72) Inventor: Marine COTTY-ESLOUS, LIBOURNE (FR)

(21) Appl. No.: 17/273,675

(22) PCT Filed: Sep. 9, 2019
(86) PCT No.: PCT/EP2019/073976

§ 371 Date: Mar. 4, 2021

Related U.S. Application Data
(60) Provisional application No. 62/728,699, filed on Sep. 7, 2018.

(51) Int. Cl. G16H 20/30; A61B 5/00; A61B 5/16; A61B 5/0205; A61B 5/021; A61B 5/0531; A61B 5/08; A61B 5/11; A61B 5/145; A61B 5/318; G06K 9/00; G16H 20/70; G06N 20/00

(52) U.S. Cl. CPC G16H 20/30; A61B 2503/40; A61B 5/165; A61B 5/7267; A61B 5/4803; A61B 5/442; A61B 5/0205; A61B 5/021; A61B 5/0531; A61B 5/0816; A61B 5/1107; A61B 5/14542; A61B 5/318; G06K 9/00302; G16H 20/70; G06N 20/00; A61B 5/4824

(57) ABSTRACT

A computer-implemented method for determining a pain treatment for a person or an animal with a pain condition, including identifying, by a processor, a level of pain being experienced by the person or animal. A method in which a level of pain experienced by the person is determined by obtaining a multimodal image or video the person, and determining the level of pain, on the basis of this multimodal image or video, via a trained Machine Learning Algorithm. The Machine Learning Algorithm is previously trained based on a training multimodal image or video of different subjects, each annotated by a benchmark pain level, determined by a biometrist and/or a health care professional based on extensive biometric data concerning the subject considered.

### CROSS-REFERENCES TO RELATED APPLICATIONS

This application claims the benefit of the International Application No. PCT/EP2019/073976, filed on Sep. 9, 2019, and of the Provisional Patent Application No. 62/728,699 filed on Sep. 7, 2018, the entire disclosures of which are incorporated herein by way of reference.

### FIELD OF THE INVENTION

The present technology relates to systems and methods of pain treatment, for example, systems and methods for determining pain treatment, or systems and methods for providing pain treatment.

### BACKGROUND OF THE INVENTION

Pain, whether acute or chronic, physical or mental pain, is a condition which is frequently treated with pharmaceutical medication. For chronic pain sufferers in particular, medication may not relieve all the symptoms of pain. Furthermore, it is not always desirable for pain sufferers to be taking pharmaceutical medication for long durations due to side effects of the pharmaceutical medication. In many cases, the pharmaceutical medication only temporarily masks the pain to the user, or worse still, the pharmaceutical medication has little or no effect on the pain.

It is thus promising to replace or to complete pharmaceutical medication by alternative treatments like digital therapeutics.

Anyhow, whatever the kind of treatment employed, it is important first to characterize the pain experienced by a person, by determining a level of pain and, if possible, complementary information relative to the pain condition of the subject, in order to adapt adequately the treatment to the person's needs (for instance, in order to adjust adequately the dosing of the medication).

Such a level of pain can be self-evaluated by the person, by indicating a value comprised between 0 (no pain) and 10 (highest conceivable pain). Such a method is fast and convenient, but a level of pain evaluated in this way turns out to be very subjective and approximate. Besides, this method provides only a value of a level of pain, with no further information regarding the pain experienced by the subject. This method cannot be employed when the person is asleep, unconscious, or unable to interact with the health care professional in charge of this pain characterization.

A pain condition of a person can be characterized in a more reliable and detailed manner by providing to the person a detailed questionnaire concerning his/her pain condition. The answers provided by the person are then analyzed and synthetized by a health care professional, such as an algologist, to determine the level of pain experienced, and additional information. But answering to such a detailed questionnaire, and analyzing the answers provided requires a lot of time, typically more than an hour.

More recently, a computer-implemented method, enabling to estimate automatically a level of pain experienced by a person by processing an image of the face of the person, has also been developed. This method is based on the FACS system (Facial Action Coding System). First, the movements, or, in other words, the deformations of the face of the person, due to muscles contraction, identified from the image of the face of the person, are decomposed (in other words, classified) into a number of predefined elementary movements, classified according to the FACS system. Then, data gathering this FACS-type information is provided to a trained neural network, which output an estimation of the pain level experienced by the person, whose face is represented in the image. The training of this neural network is based on sets of annotated training data each comprising:
a training image, representing the face of a subject; and
an annotation, constituted by a level of pain, self-evaluated by the subject.

Once the neural network has been trained, the estimation of the level of pain experienced by a person is fast and can be carried out even if the person is asleep, unconscious, or unable to interact with other people. But, this method has two major drawbacks. First, the information that is extracted from the image of the face of the person (and that is then provided as an input, to the neural network), based on the FACS system, is partial, and somehow skewed. Indeed, the predefined elementary face movements of the FACS system, which are defined for rather general facial expression classification, are somehow conventional, arbitrary. In other words, summarizing the information contained in the image of the face of the person using the general purpose FACS system, which is not designed to characterize a pain condition, causes useful information related to the pain condition to be lost, by filtering the image on a rather arbitrary basis. And in addition, the level of pain estimated by means of the neural network mentioned above finally is as subjective and approximate as a self-evaluated level of pain.

It is an object of the present technology to ameliorate at least some of the inconveniences present in the prior art. In particular, one object of the disclosed technology is to determine a level of pain experienced by a person in a fast and convenient way (so that the person's pain can be alleviated without delay), but more reliably than by self-evaluation.

### SUMMARY OF THE INVENTION

Embodiments of the present technology have been developed based on developers' appreciation of certain shortcomings associated with the existing systems for determining a treatment for alleviating, treating or reducing a pain condition of person or animal.

Embodiments of the present technology have been developed based on the developers' observation that there is no one size fits all treatment for alleviating, treating or reducing a pain condition in persons and animals suffering from the pain condition. Not only do people have a different assessment of their own pain levels, this assessment may vary from day to day. A pain treatment that works for one person may not work for another person. A pain treatment that works on one occasion for a person, may not work for the same person on another occasion. By pain condition is meant any feeling of pain, whether acute or chronic, physical or mental.

According to certain aspects and embodiments of the present technology, as defined below and in the claims, the present technology can determine tailored pain treatments for a person or animal suffering from a pain condition. In certain embodiments, the pain treatment is not only tailored for the user, but also for the particular occasion.

The disclosed technology concerns, in particular, a computer-implemented method for determining a pain treatment for a person or an animal with a pain condition, by a processor of a computer system, the method comprising:

identifying, by the processor, a level of pain being experienced by the person or animal, and

determining, by the processor, a pain treatment for the person or the animal based on the identified level of pain.

The disclosed technology concerns also a computer-implemented method for determining a level of pain experienced by a person, wherein the computer system is programmed to execute the following steps, in order to determine the level of pain experienced by the person:
obtaining a multimodal image or video, representing at least the face and an upper part of the body of the person, and comprising a voice recording of the person; and
determining the level of pain by means of a trained Machine Learning Algorithm parametrized by a set of trained coefficients, the Machine Learning Algorithm receiving input data that comprises at least the multimodal image or video, the Machine Learning Algorithm outputting output data that comprises at least the level of pain, the Machine Learning Algorithm determining the output data from the input data, on the basis of the trained coefficients;

the trained coefficients of the Machine Learning Algorithm having been previously set by training the Machine Learning Algorithm using several sets of annotated training data, each set being associated to a different subject and comprising:
training data, comprising a training multimodal image or video, representing at least the face and an upper part of the body of the subject, and comprising a voice recording of the subject considered; and
annotations associated to the training data, that comprise a benchmark pain level representative of a pain level experienced by the subject represented in the training multimodal image or video, the benchmark pain level having been determined, by a biometrist and/or a health care professional, on the basis of extensive biometric data concerning that subject, these biometric data comprising at least positions, within the training image, of some remarkable points of the face of the subject and/or distances between these remarkable points.

The extensive biometric data that is taken into account to determine the benchmark pain level considered may further comprises some or all of the following data:
skin aspect data comprising a shine, a hue and/or a texture feature of the skin of the face of the subject;
bone data, representative of a left versus right imbalance of the dimensions of at least one type of bone growth segment of the subject;
muscle data, representative of a left versus right imbalance of the dimensions of at least one type of muscle of the subject and/or representative of a contraction level of a muscle of the subject;
physiological data comprising electrodermal data, breathing rate data, blood pressure data, oxygenation rate data and/or an electrocardiogram of the subject;
corpulence data, derived from scanner data, representative of the volume or mass of all or part of the body of the subject;
genetic data, comprising data representative, for several generations within the family of the subject, of epigenetic modifications resulting from the impact of pain.

It turns out that such extensive biometric data enables to determine a very accurate and reliable level of pain, and to characterize the pain condition of the subject in a detailed manner, contrary to FACS-type archetypal face deformations, for instance.

The fact that such biometric data enables such a reliable characterization of the pain condition of a person has been discovered by the inventor by comparing the pain evaluation results obtained in this way with a classical pain evaluation based on a detailed questionnaire analyzed by a health care professional (the last one somehow playing the role of a benchmark evaluation). And it turns out that both methods lead to similar results, regarding the level of pain experienced by the person, or additional information regarding the pain condition of the person (such as the chronology of painful events experienced by the subject). For instance, when it is determined from this biometric data, in particular from the bone data mentioned above, that the person experienced a traumatic, acute pain when the person was a teenager, the answers to the detailed questionnaire provided by this person bring also to light that this person experienced a traumatic, acute pain in the past.

Besides, in a rather surprising way, it turns out that the information contained (solely) in such a multimodal image or video of a person correlates strongly with the level of pain, and with other characteristics of the pain condition experienced by the person, just as the extensive biometric data mentioned above. In other words, the information contained in such a multimodal image or video, representing the face and the upper part of the body (or a wider part of the body) of the person and including a voice recording, comprises almost as much information regarding his/her pain condition as the extensive biometric mentioned above (which is surprising as this image does not reflect directly the person's cardiac rhythm, or bone dimensions imbalance).

The disclosed technology takes advantage of this unexpected correlation between a multimodal image or video of a person and such reliable and detailed information regarding the pain condition experienced by the person. This link between the pain condition experienced by a person, and a multimodal image or video of the person, is determined by training the Machine Learning Algorithm of the computer system, as explained above. This link is stored in the computer system in the form of the coefficients that parametrize the Machine Learning Algorithm. Remarkably, once this training has been achieved, this computer system enables a characterization of the pain condition of a person both:
quickly (capturing an image or video of the face and upper part of the body of a person and recording his/her voice, and then processing this data by means of the Machine Learning Algorithm can be achieved quickly, typically in a few seconds); and
as reliably and extensively as if the pain condition of the person had been characterized using the long-to-achieve classical method of answering to a detailed questionnaire, or as if it had been characterized by gathering directly the extensive biometric data regarding the person, and by deriving a level of pain from this data (which takes also a lot of time, typically more than an hour).

The annotations associated to the different multimodal training images or videos employed to train the Machine Learning Algorithm may comprise, in addition to the benchmark pain level determined the biometrist/heath care professional, temporal features relative to the pain experienced by the subject represented in the training image considered, these temporal features specifying, for instance, whether the pain experienced by the subject is chronic or acute, and/or whether the subject had already experienced pain in the past. Such temporal features are determined by a biometrist/heath care professional, from the extensive biometric data mentioned above, when annotating the training data. In this a case, the output data of the Machine Learning Algorithm comprises also such temporal/chronological information regarding the pain experienced by the person. This is very interesting, as such information cannot be readily and quickly obtained, contrary to the multimodal image or video mentioned above.

The annotations associated with the different training images employed to train the Machine Learning Algorithm may also comprise, in addition to the benchmark pain level determined the biometrist/heath care professional (from the extensive biometric data mentioned above), some or all of the extensive biometric data mentioned above. In this case, the output data of the Machine Learning Algorithm comprises also some or all of this extensive biometric data. Which means that the computer system is then able to derive some or all of this biometric data (such as the bone, muscle, or physiological data mentioned above), from the multimodal image or video of the subject. Again, this is very interesting, as such data cannot be readily and quickly obtained, contrary to a multimodal image or video of a person.

As one may appreciate, the method for determining a pain level that has been presented above can be achieved without resorting to an identification, within the multimodal image or video of the person, of predefined, conventional types of facial movements such the ones of the FACS classification. The information loss and bias caused by such a FACS-type features extraction is thus advantageously avoided.

In the context of the present specification, unless expressly provided otherwise, a computer system may refer, but is not limited to, an “electronic device”, an “operation system”, a “system”, a “computer-based system”, a “controller unit”, a “control device” and/or any combination thereof appropriate to the relevant task at hand.

In the context of the present specification, unless expressly provided otherwise, the expression “computer-readable medium” and “memory” are intended to include media of any nature and kind whatsoever, non-limiting examples of which include RAM, ROM, disks (CD-ROMs, DVDs, floppy disks, hard disk drives, etc.), USB keys, flash memory cards, solid state-drives, and tape drives.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

Implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is a schematic illustration of a system for determining a treatment for pain, in accordance with certain embodiments of the present technology;

FIG. 2 is a computing environment of the system of FIG. 1, according to certain embodiments of the present technology;

FIG. 3 represents schematically steps of a method for determining a level of pain according to the disclosed technology;

FIG. 4 represents schematically a training phase of a machine-learning algorithm configured to determine a level of pain experienced by a person.

It should be noted that, unless otherwise explicitly specified herein, the drawings are not to scale.

### DETAILED DESCRIPTION OF THE PREFERRED EMBODIMENTS

Certain aspects and embodiments of the present technology are directed to systems 100 and methods 200 for determining a treatment for pain. Certain aspects and embodiments of the present technology are directed to systems 100 and methods 200 for providing the treatment for pain.

Broadly, certain aspects and embodiments of the present technology comprise computer-implemented systems 100 and methods 200 for determining a treatment for pain which minimizes, reduces or avoids the problems noted with the prior art. Notably, certain embodiments of the present technology determine a treatment plan for pain which is effective and which is also personalized.

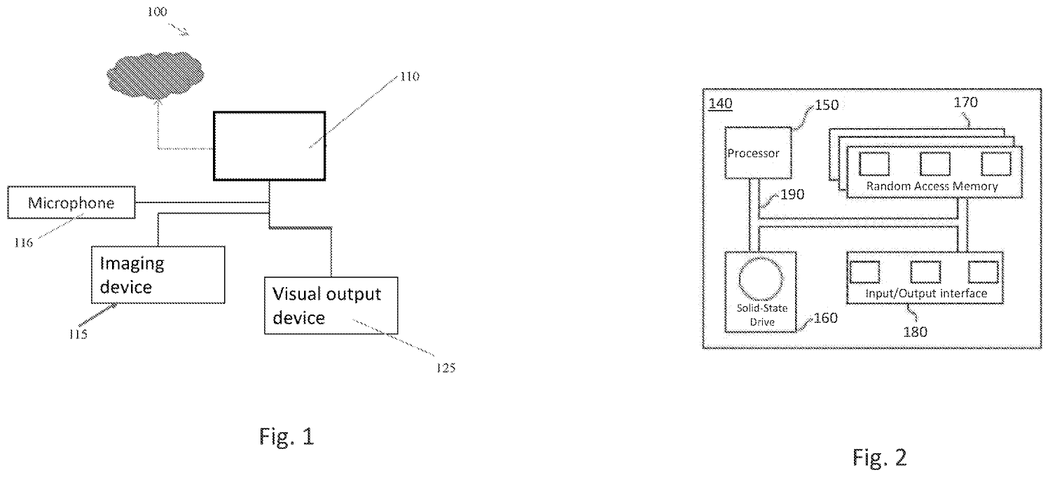

Referring to FIG. 1, there is shown an embodiment of the system 100 which comprises a computer system 110 operatively coupled to an imaging device 115 for imaging a face of a user of the system 100. Optionally, the system 100 includes one or more of a visual output device 120 for providing visual output to the user for providing sensory output to the user.

The user of the system can be any person or animal requiring or needing pain diagnosis and/or treatment. The user may be an adult, a child, a baby, an elderly person, or the like. The user may have an acute pain or a chronic pain condition.

The computer system 110 is arranged to send instructions to one or more of the visual output device, the speaker, and the haptic device, to cause them to deliver visual output, sound output or vibration output, respectively. The computer system 110 is arranged to receive visual data from the imaging device. Any one or more of the imaging device, the visual output device, the speaker, and the haptic device may be integral with one another.

In certain embodiments, the computer system 110 is connectable to one or more of the imaging device 115, the visual output device 120, the speaker 125, and the haptic device 130 via a communication network (not depicted). In some embodiments, the communication network is the Internet and/or an Intranet. Multiple embodiments of the communication network may be envisioned and will become apparent to the person skilled in the art of the present technology. The computer system 110 may also be connectable to a microphone 116, so that the voice of the person, whose pain is to be treated, can be recorded and then processed by the computer system.

Turning now to FIG. 2, certain embodiments of the computer system 110 have a computing environment 140. The computing environment 140 comprises various hardware components including one or more single or multi-core processors collectively represented by a processor 150, a solid-state drive 160, a random access memory 170 and an input/output interface 180. Communication between the various components of the computing environment 140 may be enabled by one or more internal and/or external buses 190 (e.g., a PCI bus, universal serial bus, IEEE 1394 “Firewire” bus, SCSI bus, Serial-ATA bus, ARINC bus, etc.), to which the various hardware components are electronically coupled.

The input/output interface 180 allows enabling networking capabilities such as wire or wireless access. As an example, the input/output interface 180 comprises a networking interface such as, but not limited to, a network port, a network socket, a network interface controller and the like. Multiple examples of how the networking interface may be implemented will become apparent to the person skilled in the art of the present technology. For example, but without being limiting, the networking interface 180 may implement specific physical layer and data link layer standard such as Ethernet™, Fiber Channel, Wi-Fi™ or Token Ring. The specific physical layer and the data link layer may provide a base for a full network protocol stack, allowing communication among small groups of computers on the same local area network (LAN) and large-scale network communications through routable protocols, such as Internet Protocol (IP).

According to implementations of the present technology, the solid-state drive 160 stores program instructions suitable for being loaded into the random access memory 170 and executed by the processor 150 for executing methods 400 according to certain aspects and embodiments of the present technology. For example, the program instructions may be part of a library or an application.

In this embodiment, the computing environment 140 is implemented in a generic computer system which is a conventional computer (i.e., an “off the shelf” generic computer system). The generic computer system is a desktop computer/personal computer, but may also be any other type of electronic device such as, but not limited to, a laptop, a mobile device, a smart phone, a tablet device, or a server.

In other embodiments, the computing environment 140 is implemented in a device specifically dedicated to the implementation of the present technology. For example, the computing environment 140 is implemented in an electronic device such as, but not limited to, a desktop computer/personal computer, a laptop, a mobile device, a smart phone, a tablet device, a server. The electronic device may also be dedicated to operating other devices, such as the laser-based system, or the detection system.

In some alternative embodiments, the computer system 110 or the computing environment 140 is implemented, at least partially, on one or more of the imaging device, the speaker, the visual output device, the haptic device. In some alternative embodiments, the computer system 110 may be hosted, at least partially, on a server. In some alternative embodiments, the computer system 110 may be partially or totally virtualized through a cloud architecture.

The computer system 110 may be connected to other users, such as through their respective medical clinics, therapy centers, schools, institutions, etc. through a server (not depicted).

In some embodiments, the computing environment 140 is distributed amongst multiple systems, such as one or more of the imaging device, the speaker, the visual output device, and the haptic device. In some embodiments, the computing environment 140 may be at least partially implemented in another system, as a sub-system for example. In some embodiments, the computer system 110 and the computing environment 140 may be geographically distributed.

As persons skilled in the art of the present technology may appreciate, multiple variations as to how the computing environment 140 is implemented may be envisioned without departing from the scope of the present technology.

The computer system also includes an interface (not shown) such as a screen, a keyboard and/or a mouse for allowing direct input from the user.

The imaging device is any device suitable for obtaining image data of the face of the user of the system. In certain embodiments, the imaging device is a camera, or a video camera. The computer system 110 or the imaging device is arranged to process the image data in order to distinguish various facial features and expressions which are markers of pain, for example, frown, closed eyes, tense muscles, pursed mouth shape, creases around the eyes, etc. Facial recognition software and image analysis software may be used to identify the pain markers. In certain embodiments, the image data and the determined pain markers are stored in a database.

The visual output device is arranged to present visual data, such as colors, images, writing, patterns, etc. to the user, as part of the pain treatment. In certain embodiments, the visual output device is a screen. In certain embodiments, the visual output device is a screen of the user's smartphone. In certain embodiments, the visual output device may be integral with the imaging device.

The system may also include a virtual reality headset for delivering cognitive therapy through a virtual reality experience.

The system may also include a gaming console for delivering cognitive therapy through a gaming experience.

Referring now to the method, broadly, certain embodiments of the present method comprise methods for determining a pain treatment for the user, the method comprising:

identifying a level of pain being experienced by the user, and

determining a pain treatment for the user based on the identified level of pain.

### Identifying the Level of Pain

The level of pain can be identified through the computer system obtaining image data of the face of the user, and from the image data obtaining facial markers of the level of pain of the user.

In certain embodiments, optionally, the computer system also obtains direct user input of their pain through answers to questions posed by the computer system. These can be predetermined questions, for which answers are graded according to different levels of pain.

In certain embodiments, optionally, the computer system has access to other data about the user which can help to identify the pain level. The other data can include one or more of: medical records, previous pain data, medication data, and other measured or sensed data about the user's physiology, mental state, behavioral state, emotional state, psychological state, sociological state, and cultural aspects.

The computer system, based on one or more of the facial markers, user direct responses, and other measured or sensed data about the user's physiology or mental state (collectively referred to as “pain inputs”), determines the level of pain being experienced by the user. In certain embodiments, the determined level of pain is objective. In certain embodiments, the determined level of pain is at least partially objective.

The determination of the level of pain may comprise the computer system cross-referencing the pain inputs with data in a look-up table in which the pain inputs, individually and in combination, are identified and linked to pain levels.

As described below, the determination of the level of pain comprises the computer system implementing a trained Machine Learning Algorithm (MLA) to provide the determined level of pain.

The machine-learning algorithm, implemented by the computer system 100, may comprise, without being limitative, a non-linear regression, a linear regression, a logistic regression, a decision tree, a support vector machine, a naïve bayes, K-nearest neighbors, K-means, random forest, dimensionality reduction, neural network, gradient boosting and/or adaboost MLA. In some embodiments, the MLA may be re-trained or further trained by the computer system 110 based on the data collected from the user or from sensors or other input devices associated with the user.

In certain embodiments of the present method, this can provide an objective, or at least partially objective, indicator of the pain of the user.

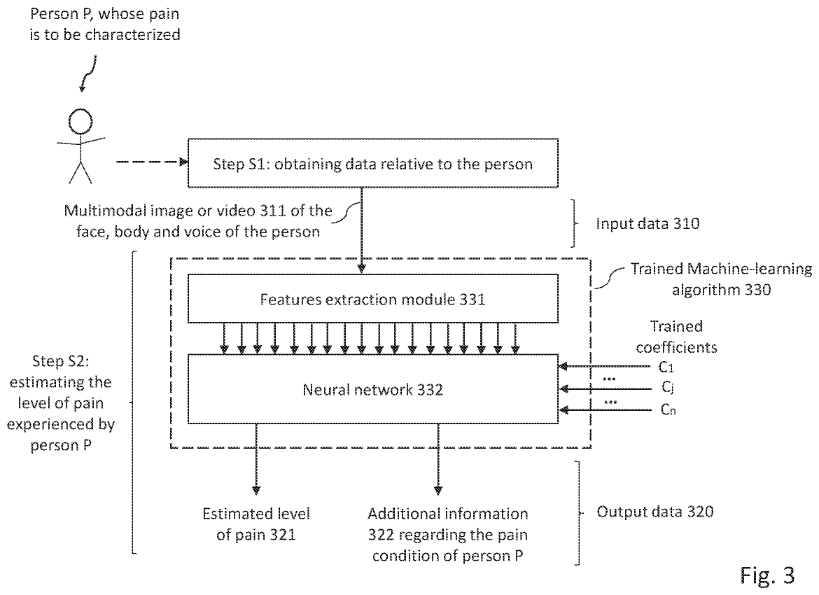

FIG. 3 represents some steps of a method for determining a level of pain experienced by a person P, based on such a machine-learning algorithm. This method comprises:

a step S1, of obtaining input data 310 to be transmitted, as an input, to the machine-learning algorithm 330; and

a step S2, of determining the level of pain 321 experienced by the person P, as well as addition information 322 regarding the pain condition experienced by that person.

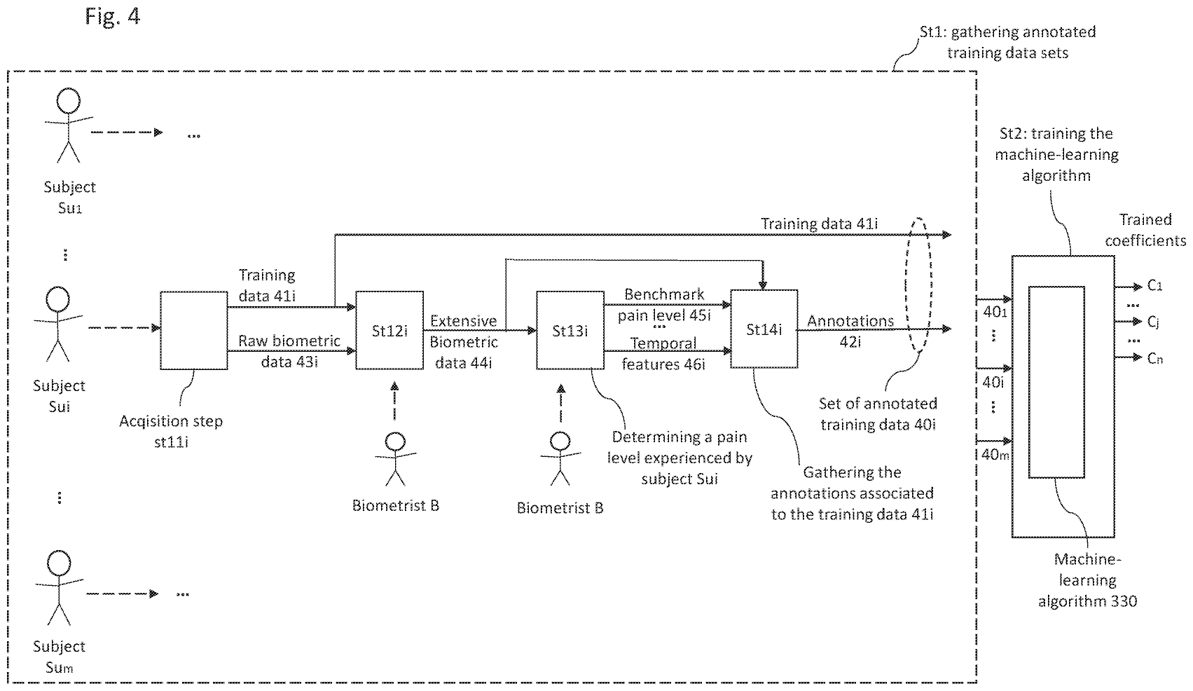

In step S1, an image, or a video gathering several successive images of the face and upper part of the body of the person P are acquired by means of the imaging device 115. A sound recording of the voice of the person is also acquired, by means of the microphone 116. In other words, in step S1, a multimodal image or video 311 representing the face and upper part of the body of the person, either statically (in the case in which a single instantaneous image is acquired) or dynamically (in the case of a video) is acquired, the multimodal image or video 311 comprising also a recording of the voice of the person. This ensemble of data is multimodal in that it comprises both facial, postural and vocal information relative to the person. The data acquired in step S1 are then transmitted to the machine learning algorithm 330. In the particular embodiment of FIG. 3, the machine learning algorithm 330 may comprises a feature extraction module 331, configured to extract key features from the input data acquired in step S1, such as a typical voice tone, in order to reduce the size of the data. In the particular embodiment of FIG. 3, the machine learning algorithm 330 may comprises a feature extraction module 331, configured to extract key features from the input data acquired in step S1, such as a typical voice tone, in order to reduce the size of the data. The features extracted by this module may comprise the facial markers mentioned above, at the beginning of the section relative to the estimation of the level of pain. Still, it will be appreciated that the features extraction employed here is achieved without resorting to an identification, within multimodal image or video 311, of predefined, conventional types of facial movements such the ones of the FACS classification. The features extracted by this module are then transmitted to a neural network 332, which determines output data, that comprises an estimation of the level of pain 321 experienced by the person, and additional information 322 regarding the pain condition of that person. This output data is determined on the basis of a number of trained coefficients C1, . . . Cj, . . . Cn, that parametrize the neural network. These trained coefficients C1, . . . Cj, . . . Cn are set during a training phase described below (with reference to FIG. 4).

The expression “neural network” refers to a complex structure formed by a plurality of layers, each layer containing a plurality of artificial neurons. An artificial neuron is an elementary processing module, which calculates a single output based on the information it receives from the previous neuron(s). Each neuron in a layer is connected to at least one neuron in a subsequent layer via an artificial synapse to which a synaptic coefficient or weight (which is one of the coefficients C1, . . . Cj, . . . Cn mentioned above) is assigned, the value of which is adjusted during the training step. It is during this training step that the weight of each artificial synapse will be determined from annotated training data.

In the embodiment described here, the additional information 322 regarding the pain condition experienced by person P comprises temporal features, that specify whether the pain experienced by the person is chronic or acute, and/or whether the person had already experienced pain in the past. The additional information 322 comprises also inferred biometric data concerning the person P, this inferred biometric data comprising here:
bone data, representative of a left versus right imbalance of the dimensions of some types of bone growth segment of that person P;
muscle data, representative of a left versus right imbalance of the dimensions of at least one type of muscle of the person and/or representative of a contraction level of a muscle of the person;
physiological data comprising electrodermal data, breathing rate data, blood pressure data, oxygenation rate data and/or cardiac activity data of the person;
corpulence data, representative of the volume or mass of all or part of the body of the person;
genetic data, comprising data representative, for several generations within the family of the person, of epigenetic modifications resulting from the impact of pain.

This biometric data is inferred in that it is not directly sensed (not directly acquired), but derived by the machine-learning algorithm 330 from the input data 310 mentioned above.

The machine learning algorithm of FIG. 3 is also configured so that the output data further comprises data representative of the condition of the person, specifying whether the person is tired or not, and/or whether the person is stressed, or relaxed,

Though FIG. 3 shows just one neural network, it will be appreciated that a machine-learning algorithm comprising more than one neural network could be employed, according to the disclosed technology.

FIG. 4 represents some steps of the training of the machine-learning algorithm 330 of FIG. 3. This training process comprises:
a step St1, of gathering several sets of annotated training data, 401, . . . ,40j, . . . ,40m, associated respectively to the different subjects Su1, . . . Sui, . . . Sum; and
step St2, of setting the coefficients C1, . . . Cj, . . . Cn of the Machine Learning Algorithm 330 by training the Machine Learning Algorithm 330 on the basis of the sets of annotated training data 401, . . . ,40j, . . . ,40m previously gathered.

In the embodiment described here, each set of annotated training data, 40i, is obtained, inter alia, by executing the following sub-steps:
St11i: acquiring training data 41i associated to subject Sui, this data comprising a multimodal training image or video representing the face and upper part of the body of the subject Sui along with a recording of the voice of subject Sui and obtaining raw biometric data 43i relative to subject Sui, such as a radiography of his/her skeleton, or such as a raw, unprocessed electrocardiogram (the sensed data about the user's physiology, mentioned previously at the beginning of the section relative to identification of the level of pain, may correspond, for instance, to these raw biometric data);
St12i: determining extensive biometric data 44i relative to subject Sui, from the training data 41i and raw biometric data 43i previously acquired, this determination being carried on by a biometrist B and/or a health care professional;
St13i: determining a benchmark pain level 45i, representative of a level of pain experienced by subject Sui, and determining temporal, chronological features 46i, regarding the pain condition experienced by subject Sui, these determinations being carried on the biometrist B and/or health care professional mentioned above;
St14i: obtaining the set of annotated training data 40i by gathering together the training data 41i associated to subject Sui and annotations 42i associated to this training data 41i, these annotations comprising the benchmark pain level 45i and the temporal, chronological features 46i, determined in step St13i, and part or all of the extensive biometric data 44i determined in step St12i.

The data type of the training data 41i acquired in step St11i is the same as the data type of the input data 310, relative to the person P whose pain condition is to be characterized, received by the machine-learning algorithm 330 once it has been trained (the training data 41i and the input data 310 contain the same kind of information). So, here, the training data 41i comprises also one or more images of the upper part of the body of subject Sui, and a sound recording of the voice of subject Sui.

The extensive biometric data 44i determined in step St12i comprises at least positions, within the training image acquired in step St11i, of some remarkable points of the face of the subject and/or distances between these remarkable points. The expression “remarkable point” is understood to mean a point of the face that can be readily and reliably (repeatedly) identified and located within an image of the face of the subject, such as one of the lips commissure, an eye canthus, an extremity of an eyebrow, or the center of the pupil of an eye of the subject. The extensive biometric data 44i comprise also posture-related data, derived from the image or images of the upper part of the body of the subject. This posture-related data may specify whether the subject's back is bent or straight, or whether his/her shoulders are humped or not, symmetrically or not.

In the embodiment described here, the extensive biometric data 44t comprises also the following data:
skin aspect data comprising a shine, a hue and/or a texture feature of the skin of the face of the subject (for instance a texture feature representative of the more or less velvety aspect of the skin of the face of the subject);
bone data, representative of a left versus right imbalance of the dimensions of at least one type of bone growth segment of the subject;
muscle data, representative of a left versus right imbalance of the dimensions of at least one type of muscle of the subject and/or representative of a contraction level of a muscle of the subject;
physiological data comprising electrodermal data, breathing rate data, blood pressure data, oxygenation rate data and/or an electrocardiogram of the subject;
corpulence data, derived from scanner data, representative of the volume or mass of all or part of the body of the subject;
genetic data, comprising data representative, for several generations within the family of the subject, of epigenetic modifications resulting from the impact of pain.

The temporal, chronological features 46i, determined during step St13i specify whether the pain experienced by the person is chronic or acute, and/or whether the person had already experienced pain in the past. Besides, in step St13i, the biometrist B and/or health care professional determines also, from the extensive biometric data 44i mentioned above, data relative to the condition of the subject, these data specifying whether the subject Sui is tired or not, and whether he/she is stressed, or relaxed. And here, the annotations 42i comprise this data, relative to the condition of the subject, in addition to the benchmark pain level 45i to the temporal, chronological features 46i, and to the extensive biometric data 41i mentioned above.

In the particular embodiment described here, the data type of the annotations 42i is thus the same as the data type of the output data 320 of the machine-learning algorithm 330, that is to say, these two data contain the same kind of information.

The process described above is repeated for each subject Su1, . . . Sui, . . . Sum. And once the sets of annotated training data, 401, . . . ,40j, . . . ,40m, associated to these different subjects have been gathered, the coefficients C1, . . . Cj, . . . Cn of the Machine Learning Algorithm 330 are set, by training the Machine Learning Algorithm 330 on the basis of these sets of annotated training data.

Determining the pain treatment for the user based on the determined level of pain.

In certain embodiments, the determined pain treatment is the provision of one or more types of sensory signals to the user. Sensory signals include, but are not limited to

visual signals (from the visible range of the electromagnetic spectrum).

Visual signals include colors, individually or in combination, images, patterns, words, etc., having an appropriate wavelength, frequency and pattern for treatment of pain, either alone or in combination with other sensory signals.

By appropriate to the treatment of pain is meant that the sensory signal provides either an endomorphic response in the user, and/or oxytocin production in the user.

In certain embodiments, the determined pain treatment further comprises providing cognitive therapy before, during or after providing the sensory signals to the person with the pain condition. In this respect, the method of determining the pain treatment also includes determining whether to provide cognitive therapy, and the type and duration of the cognitive therapy.

In certain embodiments, the determined pain treatment further comprises the manner of providing the pain treatment or the cognitive therapy. In this respect, the method of determining the pain treatment also includes determining the manner of providing the pain treatment or the cognitive therapy, and the type and duration of the cognitive therapy and the pain treatment. The manner of providing the pain treatment and/or the cognitive therapy includes one or more of a virtual reality experience, a gaming experience, a placebo experience, or the like.

As already mentioned, the pain treatment is determined based on the level of pain experienced by the person, that has been previously estimated. In the case of a pharmaceutical pain treatment, for instance, the dosing or the frequency of administration of a given sedative may be chosen as high as the level of pain is high. And the case for which the pain treatment is the provision of one or more types of sensory signals to the user, a stimulation intensity or frequency associated to these sensory signals may be chosen as high as the level of pain is high. The acute or chronic nature of the pain experienced by the person, which has been identified previously, may also be taken into account to adequately choose these sensory signals (for instance, highly stimulating signals could be chosen when pain is acute, while signals stimulating drowsiness could be chosen when pain is chronic).

It should be expressly understood that not all technical effects mentioned herein need to be enjoyed in each and every embodiment of the present technology.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1-17. (canceled)

18. A computer-implemented method for determining a pain treatment for a person or an animal with a pain condition, by a processor of a computer system, the method comprising:
identifying, by the processor, a level of pain being experienced by the person or animal, and
determining, by the processor, a pain treatment for the person or the animal based on the identified level of pain.

19. The method according to claim 18, wherein the pain treatment comprises causing one or more devices to provide one or more sensory signals to the person or the animal, the one or more sensory signals having a wavelength, frequency and pattern suitable for treating, reducing, or alleviating the pain condition in the person or the animal with the pain condition.

20. The method according to claim 18, wherein the treatment, reduction or alleviation of the pain condition of the user is measured by at least one of an endomorphic response of the user or an oxytocin response of a user.

21. The method according to claim 18, wherein determining the pain treatment further comprises determining a cognitive therapy for the person or animal with the pain condition based on at least a pain level of the person or animal.

22. The method according to claim 18, wherein determining the pain treatment comprises obtaining one or more markers of pain, the one or more markers of pain including at least one of objective or subjective markers of pain, selected from: facial expressions, facial markers, direct input from the person with the pain condition, sensed data on the person' s physiology and mental state.

23. The method according to claim 22, wherein the determining the pain treatment using the one or more markers of pain comprises implementing a trained Machine Learning Algorithm, or comprises looking up associations between the one or more markers of pain and pain treatments.

24. The method according to claim 18, for determining the pain treatment for said person, wherein the computer system is programmed to execute the following steps, in order to determine the level of pain experienced by the person:
obtaining a multimodal image or video, representing at least a face and an upper part of a body of the person, and comprising a recording of a voice of the person; and
determining said level of pain by means of a trained Machine Learning Algorithm parametrized by a set of trained coefficients, the Machine Learning Algorithm receiving input data that comprises at least said multimodal image or video, the Machine Learning Algorithm outputting output data that comprises at least said level of pain, the Machine Learning Algorithm determining said output data from said input data, on the basis of said trained coefficients;
the trained coefficients of the Machine Learning Algorithm having been previously set by training the Machine Learning Algorithm using several sets of annotated training data, each set being associated to a different subject and comprising:
training data, comprising at least a training multimodal image or video, representing at least the face and an upper part of the body of the subject considered and comprising a recording of the voice of the subject; and
annotations associated with the training data, that comprise a benchmark pain level representative of a pain level experienced by the subject represented in the multimodal training image or video, the benchmark pain level having been determined, by at least one of a biometrist or a health care professional, based on extensive biometric data concerning that subject, these biometric data comprising at least positions, within the training image, of at least one of some remarkable points of the face of the subject or distances between these remarkable points.

25. The method according to claim 24, wherein, for each set of annotated training data, the extensive biometric data that is taken into account to determine the benchmark pain level considered further comprises at least some of the following data:
skin aspect data comprising at least one of a shine, a hue or a texture feature of the skin of the face of the subject;
bone data, representative of a left versus right imbalance of dimensions of at least one type of bone growth segment of said subject;
muscle data, representative of at least one of a left versus right imbalance of dimensions of at least one type of muscle of the subject or representative of a contraction level of a muscle of the subject;
physiological data comprising at least one of electrodermal data, breathing rate data, blood pressure data, oxygenation rate data or an electrocardiogram of the subject;
corpulence data, derived from scanner data, representative of a volume or mass of all or part of the body of the subject;
genetic data, comprising data representative, for several generations within a family of the subject, of epigenetic modifications resulting from an impact of pain.

26. The method according claim 25, wherein the annotations of each set of annotated training data further comprise at least some of the extensive biometric data, from which the benchmark pain level has been determined.

27. The method according to claim 26, wherein the output data determined by the Machine Learning Algorithm further comprises inferred biometric data concerning the person whose level of pain is determined, said biometric data comprising at least one of:
skin aspect data comprising at least one of a shine, a hue or a texture feature of the skin of the face of the person;
bone data, representative of a left versus right imbalance of the dimensions of at least one type of bone growth segment of said person;
muscle data, representative of at least one of a left versus right imbalance of the dimensions of at least one type of muscle of the person or representative of a contraction level of a muscle of the person;
physiological data comprising at least one of electrodermal data, breathing rate data, blood pressure data, oxygenation rate data or cardiac activity data relative to the person;
corpulence data, representative of the volume or mass of all or part of the body of the person; or
genetic data, comprising data representative, for several generations within the family of the person, of epigenetic modifications resulting from the impact of pain.

28. The method according to claim 24, wherein:
the output data determined by the Machine Learning Algorithm further comprises temporal features concerning the pain experienced by the person, that specify at least one of whether the pain experienced by the person is chronic or acute, or whether the person had already experienced pain in the past; and
the annotations of each set of annotated training data further comprise temporal training features relative to the pain experienced by the subject represented in the training image of the set considered, the temporal training features specifying at least one of whether the pain experienced by the subject is chronic or acute, or whether the subject had already experienced pain in the past, these temporal features having been determined on the basis of the extensive biometric data concerning the subject.

29. The method according to claim 24, wherein the determination of said output data is achieved by the Machine Learning Algorithm without resorting to an identification, within the multimodal image or video of the face of the person, of predefined, conventional types of facial movements.

30. The method according to claim 24, comprising the setting of the coefficients of the Machine Learning Algorithm, said setting comprising the following steps:
gathering the sets of annotated training data, associated respectively to the different subjects, each set being obtained by executing the following sub-steps:
acquiring the training data associated to the subject considered, that comprise the training multimodal image or video that represents at least the face and an upper part of the body of the subject, and that comprises a recording of the voice of the subject;
determining the annotations associated to the training data acquired, these annotations comprising at least the benchmark pain level representative of a pain level experienced by the subject represented in said image or video, the benchmark pain level being determined by at least one of the biometrist or the health care professional on the basis of said extensive biometric data concerning the subject; and
setting the coefficients of the Machine Learning Algorithm by training the Machine Learning Algorithm on the basis of the sets of annotated training data previously gathered.

31. A computer implemented method for treating pain of a person or an animal with a pain condition, the method being implemented by a processor of a computer system, the method comprising:
determining a pain treatment for a person, according to the method of claim 18; and
providing to the person the pain treatment previously determined by the computer system, by sending instructions to one or more devices associated with the person with the pain condition, the devices being arranged to provide one or more sensory signals to the person, at a wavelength, frequency and pattern suitable for treating, reducing, or alleviating the pain condition in the person or the animal.

32. A system for determining a pain treatment, the system comprising a computer system having a processor, the processor being arranged to perform the method of claim 18.

33. A system for determining a pain treatment, the system comprising a computer system having a processor, the processor being arranged to perform the method of claim 18, further comprising an imaging device and a microphone for acquiring the multimodal image or video of the person, the system being realized in the form of a hand-held portable electronic device.

34. A system for determining a pain treatment, the system comprising a computer system having a processor, the processor being arranged to perform the method of claim 31, comprising said one or more devices associated with the person, said one or more devices comprising one or more of a device for providing visual output or a virtual reality headset, the processor being arranged to send said instructions to said device or virtual reality headset, for providing said pain treatment to the person.
